(19) United States
(12) Patent Application Publication
(10) Pub. No.: US 2022/0288383 A1
(43) Pub. Date: Sep. 15, 2022

(54) METHODS AND DEVICES FOR TRANSDERMAL NEUROSTIMULATION TREATMENT

(71) Applicant: NEUROLIEF LTD. ,, Netanya (IL)

(72) Inventors: Amit DAR, Kfar-Hess (IL); Ron BELSON, Tel Aviv (IL); Amir COHEN, Ra'anana (IL)

(21) Appl. No.: 17/635,094

(22) PCT Filed: Aug. 18, 2020
(86) PCT No.: PCT/IB2020/057781

§ 371 Date: Feb. 14, 2022

Related U.S. Application Data
(60) Provisional application No. 62/888,497, filed on Aug. 18, 2019.

(51) Int. Cl. A61N 1/04; A61N 1/36

(52) U.S. Cl. CPC A61N 1/0456; A61N 1/0476; A61N 1/0484; A61N 1/36021; A61N 1/36025; A61N 1/36034

(57) ABSTRACT

A method for determining treatment parameter values for transdermal neurostimulation treatments applied on a user's scalp comprises setting a first set of treatment parameter values for a first electrode-array engaged with a first portion of the user's scalp for administering electrical pulses thereto, for example according to a correlation that includes a direct relationship between at least one parameter value of said first set of treatment parameter values and a corresponding at least one parameter value of a set of user-specific sensory-threshold parameter values; and setting a second set of treatment parameter values for a second electrode-array engaged with a second portion of the user's scalp for administering electrical pulses thereto, according to another correlation that includes another direct relationship between at least one parameter value of said second set of treatment parameter values and said at least one parameter value of said first set of treatment parameter values.

### CROSS-REFERENCE TO RELATED APPLICATIONS

This patent application claims the benefit of U.S. Provisional Patent Application No. 62/888,497 filed on Aug. 18, 2019, which is incorporated herein by reference in its entirety.

### FIELD OF THE INVENTION

The present invention relates to apparatus and methods for applying electrical stimulation to the head region, and particularly to methods of setting, calibrating, and confirming the reliability of, electrical parameters for treatments using electrical neurostimulation, and to devices and software applications for facilitating such calibrations.

### BACKGROUND OF THE INVENTION

The present invention relates to apparatus and methods for applying electrical stimulation to the head region for neurostimulation treatments. The disclosed methods and devices may be used for calibration of treatment parameters used in the stimulation of peripheral and cranial nerves, including calibration by the users of the neurostimulation treatments.

Peripheral and cranial nerves in the head region may be stimulated to treat various conditions such as chronic pain, migraine, tension headaches, cluster headaches, trigeminal neuralgia, occipital neuralgia, fibromyalgia, depression, post-traumatic stress disorder (PTSD), anxiety, stress, bipolar disorder, schizophrenia, obsessive compulsive disorder (OCD), insomnia, epilepsy, attention deficit disorder (ADD), attention deficit hyperactivity disorder (ADHD), Parkinson's disease, Alzheimer's disease, obesity, multiple sclerosis, stroke and traumatic brain injury (TBI). The anatomy of peripheral and cranial nerves in the head region, such as that of the occipital and trigeminal nerves, and their projections to brainstem regions such as the locus coeruleus and nucleus raphe magnus, as well as to higher brain regions such as the thalamus and anterior cingulate cortex, may be advantageous when stimulating these nerves for treatment of such conditions.

Neurostimulation of superficial nerves in the head region, such as the occipital, supraorbital, supratrochlear, zygomaticotemporal, and auriculotemporal nerve branches, can be applied either invasively or non-invasively. Due to the challenge of transferring current through the hair, stimulation of cephalic nerves which lie under hair covered regions such as the occipital nerve (greater, lesser and third occipital branches), is typically carried out using implanted or percutaneous nerve stimulators. Such devices include electrodes that are inserted under the scalp and thus bypass the high impedance barrier formed by the hair and scalp. However, implanted nerve stimulation remains an invasive and costly procedure with a high rate of complications including infection, bleeding or fluid collection under the skin, as well as hardware-related malfunctions such as migration and breakage of the implanted leads and pulse generator failure. Transdermal stimulation of cephalic nerves such as the occipital nerve branches using non-invasive techniques is likely to achieve similar clinical benefits to those of implanted stimulators without the risks and cost associated with an invasive procedure.

Transdermal neurostimulation treatments have the promise of being carried out by the subjects of such treatments in the comforts of their own personal surroundings. It is noted, however, that even transdermal treatments can require the user-specific and personalized setting of some treatment parameters such as, for example, the current intensities and pulse durations encountered during a treatment. Moreover, electrical stimulation directed at different parts of the user's head may require differentiated treatment parameters for effective stimulation of the different nerves involved. Treatment parameters are optimally set and calibrated so as to take into account both the efficacy of treatments and the comfort level of users undergoing the treatments.

There is therefore a recognized need for, and it would be highly advantageous to have, devices and methods for effective setting and calibration of neurostimulation treatment parameters that can operated effectively by both medical professionals and users of the treatment.

Systems, apparatuses and methods for delivery of neurostimulation treatments are disclosed in Applicant's U.S. Pat. Nos. 9,433,774 and 9,872,979, and in Applicant's co-pending U.S. patent application Ser. No. 15/510,067, filed on Sep. 16, 2015, and published as US 20170296121; 16/070,563, filed on Jan. 26, 2017, and published as US 20190022372; and Ser. No. 16/093,094, filed on Apr. 9, 2017, and published as US 20190117976; each of the foregoing patents and co-pending patent applications is incorporated herein by reference in its entirety.

### SUMMARY OF THE INVENTION

A method is disclosed, according to embodiments, for determining user-specific treatment parameter values for a multi-channel transdermal neurostimulation treatment applied at multiple locations on a user's scalp. The method comprises: (a) setting a first set of treatment parameter values for a first electrode-array in engagement with a first portion of the user's scalp for administering electrical pulses thereto, according to a first correlation that includes a first direct relationship between at least one parameter value of said first set of treatment parameter values and a corresponding at least one parameter value of a set of user-specific sensory-threshold parameter values; and (b) setting a second set of treatment parameter values for a second electrode-array in engagement with a second portion of the user's scalp for administering electrical pulses thereto, according to a second correlation that includes a second direct relationship between at least one parameter value of said second set of treatment parameter values and said at least one parameter value of said first set of treatment parameter values.

In some embodiments, each respective set of treatment parameter values can include values for at least two of the following: (i) an electrical charge parameter having values of total electric charge for a given time period, where the given time period is an integer multiple of an inverse of electrical pulse frequency, (ii) a current-intensity parameter, and (iii) a pulse duration parameter and a frequency parameter, or an arithmetic combination thereof.

In some embodiments, either (i) said first portion of the scalp is an anterior portion and said second portion of the scalp is a posterior portion, or (ii) said first portion of the scalp is a posterior portion and said second portion of the scalp is an anterior portion.

In some embodiments, said second direct relationship can be based in part on a relationship between respective electrode surface-areas of said first and second electrode arrays.

In some embodiments, values of the set of user-specific sensory-threshold parameter values are based on a first user input received during application of a series of electrical pulses to a single one of the first and second portions of the user's scalp.

In some embodiments, said first and second correlations can be linear relationships based on respective empirical correlations.

In some embodiments, it can be that according to said first direct relationship, an electrical charge parameter value of said first set of treatment parameter values is equal to an electrical charge parameter value of said set of user-specific sensory-threshold parameter values multiplied by A, where A is not less than 1.1 and not greater than 3.2. In some embodiments, it can be that A is not less than 1.4 and not greater than 2.0. In some embodiments, it can be that A is not less than 1.3 and not greater than 1.9.

In some embodiments, it can be that according to said second direct relationship, an electrical charge parameter value of said second set of treatment parameter values is equal to an electrical charge parameter value of said first set of treatment parameter values multiplied by B, where B is not less than 1.1 and not greater than 3.8. In some embodiments, it can be that B is not less than 1.4 and not greater than 3.4. In some embodiments, it can be that B is not less than 1.8 and not greater than 3.0.

In some embodiments, the method can additionally comprise: calibrating treatment thresholds, wherein the calibrating includes: (i) activating a transdermal neurostimulation calibration session using said first and second set of treatment parameter values as respective treatment parameter values applied to said first and second portions of the user's scalp, (ii) receiving a user-calibration input during said calibration session, and (iii) in response to receiving said user-calibration input, changing at least one value of said first and second set of treatment parameter values.

In some embodiments, said changing of said at least one value of said first and second set of treatment parameter values can include modifying at least one treatment parameter value that is not a current intensity.

In some embodiments, said changing can be constrained such that an electrical charge parameter value is constrained to being within a predetermined range. In some embodiments, said changing can be constrained such that the number of changes per calibration session is limited to a predetermined number of changes. In some embodiments, said changing can be constrained to a predetermined interval of time. In some embodiments, the time between receiving of the user-calibration input and completing the changing can be less than one second. In some embodiments, the time between receiving of the user-calibration input and completing the changing can be less than 500 msec. In some embodiments, the time between receiving of the user-calibration input and completing the changing can be less than 200 msec.

In some embodiments, the transdermal neurostimulation treatment can include anteriorly-applied electrical pulses targeting the trigeminal nerve and posteriorly-applied electrical pulses targeting the occipital nerve.

In some embodiments, said administering of electrical pulses can be by an apparatus including electrodes and mounted on the user's head, said apparatus comprising a headset.

In some embodiments, at least one of said first user input and said user-calibration input can be received via a computer software program running on an external user-operated device in electronic communication with said headset. In some such embodiments, it can be that (i) said headset comprises a on-headset user interface attached to said headset, and/or (b) at least one of said first user input and said user-calibration input is received via said on-headset user interface.

A method is disclosed, according to embodiments, for determining user-specific treatment parameter values for a multi-channel transdermal neurostimulation treatment applied at multiple locations on a user's scalp. The method comprises: (a) setting a first set of treatment parameter values for a first electrode-array in engagement with a first portion of the user's scalp for administering electrical pulses thereto; and (b) setting a second set of treatment parameter values for a second electrode-array in engagement with a second portion of the user's scalp for administering electrical pulses thereto, according to a second correlation that includes a direct relationship between at least one parameter value of said second set of treatment parameter values and said at least one parameter value of said first set of treatment parameter values.

In some embodiments, each respective set of treatment parameter values can include values for at least two of: (i) an electrical charge parameter having values of total electric charge for a given time period, where the given time period is an integer multiple of an inverse of electrical pulse frequency, (ii) a current-intensity parameter, and (iii) a pulse duration parameter and a frequency parameter, or an arithmetic combination thereof.

In some embodiments, it can be that either (i) said first portion of the scalp is an anterior portion and said second portion of the scalp is a posterior portion, or (ii) said first portion of the scalp is a posterior portion and said second portion of the scalp is an anterior portion.

In some embodiments, said direct relationship can be based in part on a relationship between respective electrode surface-areas of said first and second electrode arrays.

In some embodiments, wherein said correlation can be a linear relationship based on an empirical correlation.

In some embodiments, it can be that according to said direct relationship, an electrical charge parameter value of said second set of treatment parameter values is equal to an electrical charge parameter value of said first set of treatment parameter values multiplied by B, where B is not less than 1.1 and not greater than 3.8. In some embodiments, it can be that B is not less than 1.4 and not greater than 3.4. In some embodiments, it can be that B is not less than 1.8 and not greater than 3.0.

In some embodiments, the transdermal neurostimulation treatment can include anteriorly-applied electrical pulses targeting the trigeminal nerve and posteriorly-applied electrical pulses targeting the occipital nerve.

In some embodiments, said administering of electrical pulses can be by an apparatus including electrodes and mounted on the user's head, said apparatus comprising a headset.

A method is disclosed according to embodiments, for determining user-specific treatment parameters for a multi-channel transdermal neurostimulation treatment applied at multiple locations on a user's scalp. The method comprises: (a) providing, to the user's scalp, a first series of electrical pulses having electrical parameter values that increase at a first rate of increase, wherein said respective electrical parameter values comprise at least one of an electrical charge parameter value and a current-intensity value; (b) responsively to receiving a first user input indicating that the user has felt a dermal sensation related to the application of said first series of electrical pulses, defining, as a set of sensory-threshold parameter values, a plurality of respective electrical parameter values at which said first user input is received; (c) providing, to the user's scalp, a reliability-confirming series of electrical pulses having respective electrical parameter values that increase at a second rate of increase, wherein said respective electrical parameter values comprise at least one of an electrical charge parameter value and a current-intensity value, the providing being such that said reliability-confirming series is (i) paused before respective electrical parameter values reach said sensory-threshold parameter values, and (ii) subsequently resumed after a pause; (d) responsively to receiving, after said pause and at respective electrical parameter values within a predetermined range of values surrounding said sensory-threshold parameter values, a second user input indicating that the user has felt a dermal sensation related to the application of said reliability-confirming series of electrical pulses: confirming the reliability of said sensory-threshold parameter values; and (e) applying the multi-channel transdermal neurostimulation treatment using treatment parameter values based on said reliability-confirmed sensory-threshold parameter values.

In some embodiments, said treatment parameter values can include values for at least two of: (i) an electrical charge parameter having values of total electric charge for a given time period, where the given time period is an integer multiple of an inverse of electrical pulse frequency, (ii) a current-intensity parameter, and (iii) a pulse duration parameter and a frequency parameter, or an arithmetic combination thereof.

In some embodiments, it can be that said first series of electrical pulses is applied to a first portion of the user's scalp, and said reliability-confirming series of electrical pulses is applied to a second portion of the scalp that is different from the first portion. In some embodiments, it can be that said first series of electrical pulses and said reliability-confirming series of electrical pulses are both applied to a first portion of the user's scalp.

In some embodiments, it can be that either (i) said first portion of the scalp is an anterior portion and said second portion of the scalp is a posterior portion, or (ii) said first portion of the scalp is a posterior portion and said second portion of the scalp is an anterior portion.

In some embodiments, the transdermal neurostimulation treatment can include anteriorly-applied electrical pulses targeting the trigeminal nerve and posteriorly-applied electrical pulses targeting the occipital nerve.

In some embodiments, said providing of said first series of electrical pulses and said providing of said reliability-confirming series of electrical pulses can be by an apparatus including electrodes and mounted on the user's head, said apparatus comprising a headset.

In some embodiments, at least one of said first user input and said second input can be received via a computer software program running on an external user-operated device in electronic communication with said headset.

In some embodiments, it can be that (i) said headset comprises a on-headset user interface attached to said headset, and (ii) at least one of said first user input and said second user input is received via said on-headset user interface.

According to embodiments, a non-transitory computer-readable medium having stored therein program instructions, which when executed by one or more processors, cause the one or more processors to carry out any of the steps of any of the methods disclosed herein.

According to embodiments, a user input device for use during a transdermal neurostimulation treatment comprises a user interface; one or more processors; and a non-transitory computer-readable medium having stored therein program instructions, which when executed by said one or more processors of the user input device, cause said one or more processors to carry out any of the steps of any of the methods disclosed herein. In some embodiments, the user input device can be in electronic communication with an apparatus comprising a headset and a plurality of electrodes configured to deliver electric pulses to a user's scalp.

### BRIEF DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like functionalities, but not necessarily identical elements.

In the drawings:

FIG. 1 is a schematic block diagram of an embodiment of an inventive system for neurostimulation according to an embodiment of the teachings herein.

FIG. 2 provides a schematic, perspective view illustration of a donned, inventive headset adapted to communicate with a remote-control unit, mobile phone, and computer.

FIG. 3 is a perspective view schematic illustration of an embodiment of the inventive system of FIG. 1A in the form of a headset according to the teachings herein.

FIGS. 4-13 show flowcharts of method building blocks comprising method steps for defining, setting, testing, confirming, calibrating and applying parameters for transdermal neurostimulation, according to embodiments of the present invention.

FIGS. 14-19 show flowcharts of methods which various method steps and method building blocks of FIGS. 4-13, according to embodiments of the present invention.

### DETAILED DESCRIPTION

Systems and methods are described herein that apply electrical stimulation to the head region, for stimulation of peripheral and/or cranial nerves, transcranial stimulation of brain regions, and sensing of body parameters, while monitoring the tissue and adapting the electrical stimulation signal to the user's characteristics and to changes over time. The systems and methods ensure effective electrical stimulation while avoiding damage to scalp tissue as well as discomfort to the user, and operate in a safe and robust fashion.

The inventive methods may be applied using a head mounted construction serving as a platform for applying electrical stimulation in accordance with the inventive methods to treat various conditions such as migraine, tension type headaches, cluster headaches, trigeminal neuralgia, occipital neuralgia, chronic pain, fibromyalgia, stress, depression, post-traumatic stress disorder (PTSD), anxiety, obsessive compulsive disorder (OCD), insomnia, epilepsy, attention deficit hyperactivity disorder (ADHD), Parkinson's disease, Alzheimer's disease, obesity, multiple sclerosis, traumatic brain injury (TBI) and stroke.

The term ‘user’ as employed herein means a person who receives or is going to receive transdermal stimulation treatment using electrical stimulation. Electrical stimulation of a pure sensory nerve elicits radiation of a paresthesia sensation along the distribution of the nerve in response to applied electrical stimulation.

This disclosure deals with ‘dermal sensation’ that a user may experience in response to the application of transdermal electrical neurostimulation, and may include one or more of tingling, pricking, chilling, burning, or numbness; such dermal sensation may be equivalent to the sensation of paresthesia.

A minimum threshold at which a user feels or acknowledges feeling such a dermal sensation or paresthesia is called a ‘sensory threshold’; a sensory threshold may be particular to a user at a given time under given circumstances and conditions.

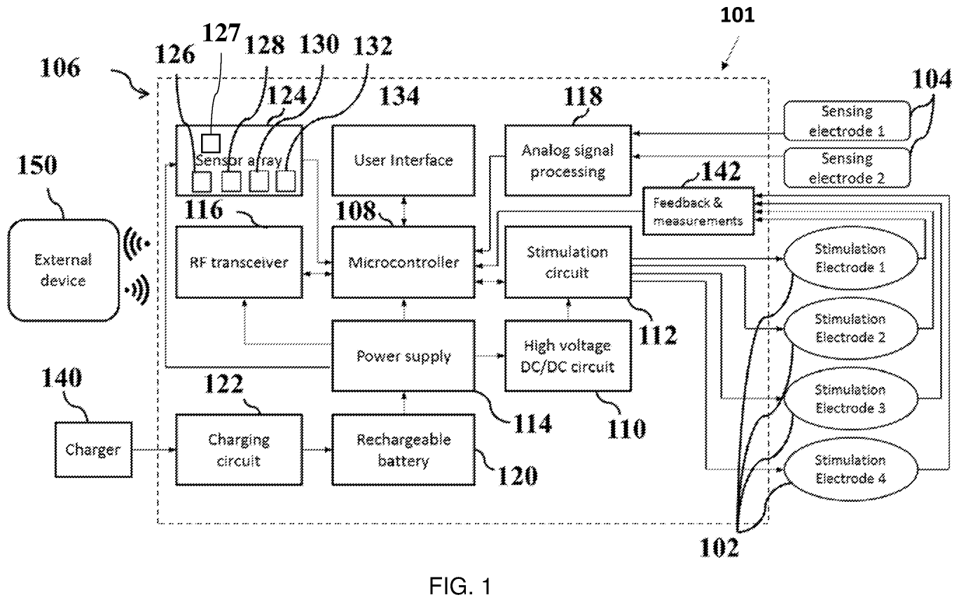

Reference is now made to FIG. 1, which is a schematic block diagram of an embodiment of an exemplary system for neurostimulation according to an embodiment of the teachings herein.

As seen, a system 101 for neurostimulation may include at least two stimulating electrodes 102, and in some embodiments may further include at least two sensing electrodes 104, both the electrodes 102 and 104 functionally associated with an electronic circuit 106. The stimulating electrodes 102 are adapted to engage the skin of the user's scalp so as to deliver current thereto as described hereinbelow. In some embodiments, one or more of sensing electrodes 104 may be adapted to engage the skin of the user, and may be configured to sense at least one electrical parameter of a body portion of the user, such as, for example, electroencephalogram (EEG), skin conductance response (SCR), impedance plethysmograph (IPG), electromyograph (EMG), and the like.

According to features of the teachings herein, system 101, and specifically the electronic circuit 106, may be suited for applying transcranial electrical stimulation using suitable methods such as Transcranial Direct Current Stimulation (tDCS), Transcranial Alternating Current Stimulation (tACS), and Transcranial Random Noise Stimulation (tRNS). Insofar as a treatment or application of electric stimulation described herein relates to a user's head or scalp, then the terms ‘transdermal’ and ‘transcranial’ may be used interchangeably. The term ‘scalp’ as used herein should be understood in the broadest sense of the term, and especially as being inclusive of the skin of the forehead, i.e., that part of a user's ‘face’ which is above the user's eyes.

As seen, the electronic circuit 106 may include any one or more of a microcontroller 108, a high voltage circuit 110, a stimulation circuit 112, an internal power supply 114, a radio-frequency (RF) transceiver 116, an analog signal processing circuit 118, a rechargeable battery 120 electrically associated with a charging circuit 122, a sensor array 124 including one or more of an accelerometer 126, a temperature sensor 128, a pressure sensor 130, and a humidity sensor 132, and a user interface 134.

In some embodiments, the electronic circuit 106 may be electrically associated with and powered by rechargeable battery 120 that is electrically connected to internal power supply 114. In some embodiments, the internal power supply 114 provides power to high voltage circuit 110, which in turn is electrically connected to stimulation circuit 112. The charging circuit 122 is electrically associated with rechargeable battery 120, and may interface with an external power supply, such as a charger 140. The high voltage circuit 110 provides to stimulation circuit 112 current with voltage in the range of 1 to 150 V. In some embodiments, the rechargeable battery 120 may be replaced by a consumable battery configured to operate in the same ranges of voltage and current.

In some embodiments, the stimulation circuit 112 receives information and/or commands from the microcontroller 108. The stimulation circuit 112 is configured to provide electrical stimulation pulses to the user's nerve tissues via the stimulation electrodes 102.

The stimulation circuit 112 may be configured to produce biphasic, charged balanced electrical pulses, mono-phasic electrical pulses, and/or direct current stimulation.

According to still further features of the described embodiments, the stimulation circuit 112 may be configured to produce electrical stimulation within an intensity range of 0-60 mA, 0-40 mA, 0-20 m, or 0-15 mA.

According to still further features of the described embodiments, the stimulation circuit 112 may be configured to produce stimulation pulses with a duration of 10-1000 μsec, 50-600 μsec, 100-500 μsec.

According to still further features of the described embodiments, the stimulation circuit 112 may be configured to produce stimulation pulses at a frequency of 1-20,000 Hz, 1-10,000 Hz, 1-500 Hz, 10-300 Hz, 10-250 Hz, 20-180 Hz, 30-180 Hz or 40-100 Hz.

In some embodiments, electronic circuit 106 may include two or more high voltage circuits (not shown) similar to circuit 112, each high voltage circuit providing current at a voltage of 1-150V, 1-120V, 1-100V, to at least two of stimulation electrodes 102. In some embodiments, electronic circuit 106 may include at least two isolated output channels (not shown), each output channel providing output to at least two of stimulation electrodes 102.

In some embodiments, the electronic circuit 106 also includes a feedback & measurements circuit 142, which collects voltage or current level information from the stimulation electrodes 102, and provides the collected information to the microcontroller 108. The microcontroller 108 uses the provided feedback to monitor and control the voltage and current levels in stimulation electrodes 102 via stimulation circuit 112. In some embodiments, the microcontroller 108 may alert the user, for example by providing an audible or tactile indication, or may halt the provision of current for stimulation in the case of an emergency or of incorrect function of the system.

In some embodiments, the microcontroller 108 may instruct the stimulation circuit 112 to output electrical current in various patterns and/or for various periods of time, and/or may instruct the stimulation circuit 112 with regards to various stimulation parameters, such as the current amplitude, pulse frequency, phase duration, and amplitude of the current output by the stimulation circuit.

In some embodiments, the microcontroller 108 may instruct the stimulation circuit 112 to provide an output signal having a different pattern for each of a plurality of activated pairs of electrodes. For example, the stimulation circuit 112 may stimulate one pair of electrodes at a pulse frequency of 50 Hz and a phase duration of 300 μsec and another pair of electrodes at a pulse frequency of 100 Hz and a phase duration of 200 μsec. In some embodiments, at any given time the microcontroller 108 may activate only one pair of electrodes, may activate a combination of electrodes, and/or may activate several electrodes simultaneously, sequentially, or alternately.

In some embodiments, some electrodes 102 may provide as output an alternating current signal, whereas other electrodes 102 may provide as output a direct current. In some embodiments, at least two electrodes 102 may alternate the type of current provided as output between alternating current and direct current.

In some embodiments, during direct current stimulation in which excitation of a certain region of the brain is determined based on the polarity of an electrode which is positioned above that region of the brain, at least one electrode 102 may be assigned by the microcontroller 108 to be the anode, or positively charged electrode, and at least one other electrode 102 may be assigned to be the cathode, or negatively charged electrode.

In some embodiments, stimulation patterns determined by or assigned by the microcontroller 108, as well as feedback data received from electrodes 102 and/or from sensing electrodes 104 may be stored in the microcontroller 108 or in a volatile or non-volatile memory (not shown) associated therewith. In some embodiments, the stored stimulation patterns may be used to create a personalized neurostimulation treatment protocol.

In some embodiments, electronic circuit 106 may be configured to receive analog signal input, such as electroencephalogram (EEG) signals, skin conductance response (SCR) signals, impedance plethysmograph (IPG) signals, electromyograph (EMG) signals, or other bio-signals, from one or more sensors, such as sensing electrodes 104, which bio-signals may be indicative of the impedance of the tissue receiving the neurostimulation signal, the charge provided to the tissue, or the like. The analog signal input received from sensing electrodes 104 may be processed by analog signal processing circuit 118, and may be transferred therefrom to microcontroller 108. In some embodiments, electronic circuit 106 may be configured to receive digital, analog, or other input from additional sensors adapted to sense the vicinity of the user or characteristics thereof. In some embodiments, one or more stimulation parameters may be altered by the microcontroller 108 due to inputs received from one or more of the additional sensors, as described hereinbelow.

In some embodiments, accelerometer 126, or any other suitable orientation sensor, may be configured to sense the angular position of the user's head or of an apparatus embodying system 101 (and particularly portions thereof engaging the user's head), and thereby may enable microcontroller 108 to identify a change in the user's and/or system's conditions and to adjust or adapt the pulse provided by stimulating electrodes 102. For example, a change in the position of the user may result in a change in the pressure applied to the electrodes, thus changing how close the electrodes are to the user's skin and consequently changing the impedance in the system and requiring adaptation of the pulse applied to the tissue via the electrodes, as described hereinbelow. In some embodiments, a vibrating structure microelectromechanical systems (MEMS) gyroscope 127 can be configured to provide angular motion information additional to motion and orientation information provided by the accelerometer 126.

In some embodiments, temperature sensor 128 may be configured to sense a temperature in the vicinity of the system 101 or of the stimulating electrodes 102, and thereby may enable microcontroller 108 to identify a change in the user's and/or system's conditions and to adjust or adapt the pulse provided by stimulating electrodes 102. For example, an increase in the temperature in the vicinity of the user or of the electrodes 102 may result in more rapid dehydration of the electrodes or of conducting material applied thereto, thus increasing the impedance in the system and requiring adaptation of the pulse applied to the tissue via the electrodes, as described hereinbelow.

In some embodiments, pressure sensor 130 may be configured to sense pressure applied to the user's head in the vicinity of electrodes 102 or pressure applied directly to electrodes 102, and thereby may enable microcontroller 108 to identify a change in the user's and/or system's conditions and to adjust or adapt the pulse provided by stimulating electrodes 102. For example, an increase in the amount of pressure applied to electrodes 102 pushing them towards the user's scalp is expected to reduce the distance between the electrodes and the scalp, and in some cases the distance between the electrode and the target nerve, thereby reducing the impedance in the system and requiring, or allowing, adaptation of the pulse applied to the tissue via the electrodes.

In some embodiments, humidity sensor 132 may be configured to sense a humidity or moisture level in the vicinity of the system 101 or of the stimulating electrodes 102, and thereby may enable microcontroller 108 to identify a change in the user's and/or system's conditions and to adjust or adapt the pulse provided by stimulating electrodes 102.

In some embodiments, user interface 134 may be configured to receive from the user an indication of the sensation the user is feeling, such as an indication of pain, an indication of discomfort, or an indication of decreased, or no, paresthesia (dermal sensation). Such an indication from the user of a change in the sensation the user feels may enable microcontroller 108 to adjust or adapt the pulse provided by stimulating electrodes 102, or to assess sensory and treatment thresholds and user-calibration of thresholds, as will be described elsewhere in this disclosure.

In some embodiments, RF transceiver 116 may enable the microcontroller 108 to communicate with an interface of an external device 150, such as a mobile phone, a tablet, a computer, or a cloud-based database, by way of radio frequency. The RF transceiver 116 may transmit digital information to and may receive digital information from the microcontroller 108, for example for personalization of the neurostimulation treatment provided by system 101. RF transceiver 116 may be operable to transmit/receive in any of the wireless communications protocols known in the art, including, but not exhaustively, Wi-fi, WLAN, PDA, VoIP, and Bluetooth.

The interface of device 150 may comprise a software application that may be downloadable from a readily accessible resource, such as from the Internet. The interface may provide to a user thereof an indication, for example by way of a display, of the status of the system 101, including, for example, information relating to active stimulation channels, stimulation intensity, active program, treatment time, battery status, and RF communication status, as well as various alerts such as alerts relating to electrode contact quality and to proper or improper system alignment on the head. Additionally, the interface may provide to the user, for example by way of a display, usage logs and/or reports, such as information relating to daily stimulation time, stimulation parameters which were used during stimulation, and treatment programs which were used. The interface may also display, or otherwise provide, to the user raw or processed information received from sensors included in or associated with the apparatus.

In some embodiments, the system may be controlled remotely via the interface of external device 150. For example, the external interface may enable a user thereof to activate or turn off the system, start or pause stimulation, adjust the stimulation intensity for one or more channels, and select a treatment program. In some embodiments, information collected by the microprocessor 108 may be transmitted, via the external interface, to a remote location, such as a cloud-based portal, where the information may be stored or may be analyzed and/or monitored.

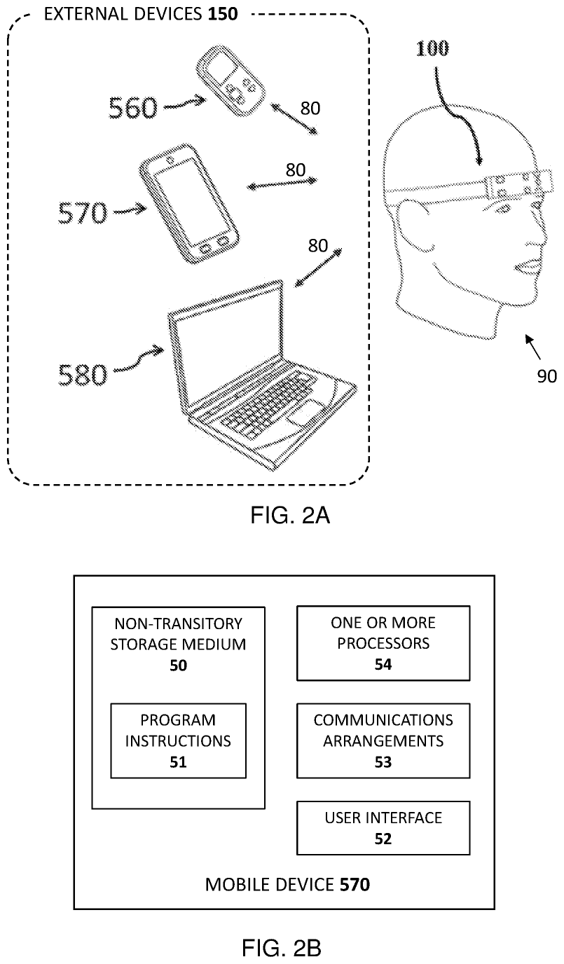

We now refer to FIG. 2A, which illustrates several components of a neurostimulation treatment system. A headset 100 is mounted on the head 90 of a user. In some embodiments, headset 100 may be configured to communicate wirelessly with one or more external devices 150 using communications channel(s) 80. Any communications channel 80 may utilize any combination of direct device-to-device communications (e.g., by wi-fi or Bluetooth or IR or any other wireless device-to-device communication or wired communication) and indirect communications (e.g., routed through servers and/or routers and/or other local network devices and Internet devices).

An example of an external device 150 is remote control 560, which may be used by the user to send commands and other inputs to headset 100. Remote control 560 may also present various visual and audio indications for the user regarding the status of headset 100. Additionally or alternatively, headset 100 may be configured to wirelessly communicate with a mobile phone 570. The mobile phone interface may be used to present various data sent wirelessly by headset 100, for example, visual and audio indications regarding the status of headset 100 and usage logs. As shown in the block diagram of an exemplary handheld device such as a mobile phone 570 in FIG. 2B, mobile phone 570 includes a non-transitory computer-readable storage medium 50 on which are stored program instructions 51 for execution by the one or more processors 54 of the mobile phone 570. The program instructions 51 may cause the mobile phone 570 and/or the headset 100 to control a neurostimulation treatment session, including, for example, to determine treatment protocol parameters before a treatment session, to capture user instructions and other inputs, and/or to determine treatment protocol parameters. The mobile phone 570 of FIG. 2B additionally includes communications arrangements 53 for communicating with headset 100 (inter alia), and a user interface 52 which may include a touch screen. The user interface 52 can be used for any manner of interactions between user and the software program comprising program instructions 51, and the user interface 52 may be used to present various data sent wirelessly by headset 100, such as visual and audio indications regarding the status of headset 100 and usage logs.

Still referring to FIG. 2A, headset 100 may additionally or alternatively be configured to wirelessly communicate with laptop/PC 580. Like mobile phone 570 Laptop/PC 580 may be configured with a non-transitory computer-readable storage medium on which are stored program instructions 51.

Any one or more of the illustrated ‘user input devices’ 150 of FIG. 2A (mobile phone 570, remote control 560, and PC/laptop 580) may be used interchangeably in carrying out the methods disclosed herein. Headsets 100 suitable for carrying out the methods disclosed herein include any of the headsets disclosed in Applicant's U.S. Pat. Nos. 9,433,774 and 9,872,979, and in Applicant's co-pending U.S. patent application Ser. No. 15/510,067, filed on Sep. 16, 2015, and published as US 20170296121; Ser. No. 16/070,563, filed on Jan. 26, 2017, and published as US 20190022372; and Ser. No. 16/093,094, filed on Apr. 9, 2017, and published as US 20190117976.

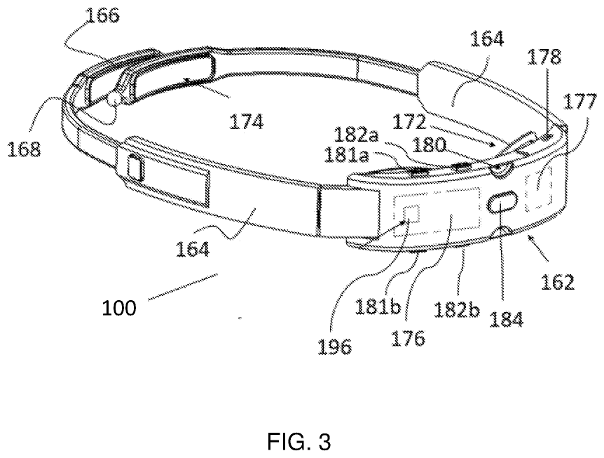

Reference is now made to FIG. 3, which is a perspective view schematic illustration of a non-limiting exemplary embodiment of a headset 100 implementing the system 101 of FIG. 1. As seen, a headset 100 may be configured to include an anterior member 162 connected to a pair of flexible arm members 164, which may also be called interim members, each terminating in a posterior member 166. Anterior member 162, flexible arm members 164, and posterior members 166 together form the headset's body. In some embodiments, electrode systems 172 may comprise anterior electrodes adapted to be located at the supraorbital region of the head over the trigeminal nerve branches for stimulation thereof, or may be electrodes suitable for transcranial stimulation of the frontal and prefrontal region of the brain. In some embodiments, electrode systems 174 may comprise posterior electrodes adapted to be located at the occipital region of the head over the occipital nerve branches for stimulation thereof, or may be electrodes suitable for transcranial stimulation of the occipital region of the brain.

It will be appreciated that headset 100 may include additional electrodes having similar structure and/or functionality to those of electrode systems 172 and 174. It is further appreciated that electrode systems 172 and/or 174 may be obviated, or moved to other locations on headset 100, as suitable for stimulating specific nerves or nerve sets, or specific brain regions. For example, electrode systems 174 may be moved to be along the flexible arm members 164. As another example, the headset 100 may include only a single pair of electrode systems located on arm members 164, which electrodes may be configured to be positioned, when the headset is donned, under the hair, while electrode systems 172 and 174 may be obviated. In some embodiments, electrodes can be directed to stimulating nerves located in lateral regions of the brain, additionally or alternatively to stimulation of anterior and/or posterior nerves.

Anterior member 162 may be configured to contain an electronic circuit 176, similar to electronic circuit 106 of FIG. 1, which may be configured to be electrically coupled by conductive wires (not shown) to a power source 177, such as a battery similar to battery 120 of FIG. 1, and to electrodes systems 172 and 174. In some embodiments, at least a portion of the conductive wires extends to posterior electrode systems 174 via arm members 164.

In some embodiments, the electronic circuit 176 and/or the battery 177 may be external to headset 100, and/or may communicate remotely with headset 100.

As discussed hereinabove with reference to FIG. 1, the electronic circuit 176 may include a stimulation circuit, a microprocessor, a charging circuit and a user interface.

In some embodiments, headset 100 may be configured to connect to an external electronic circuit and/or stimulation circuit, and thereby to transfer electrical current from an external stimulator to the electrode systems 172 and/or 174. In some embodiments, headset 100 may be configured to connect to at least one external electrode that may be located at various areas of the body. In some embodiments, headset 100 may be configured to connect to an external electronic circuit and processor in order to transfer signals from sensors disposed on the headset 100 to the external processor.

In some embodiments, battery 177 may be disposed within anterior member 162, and may be recharged by plugging a charger into charging port 178 located, according to certain embodiments, on anterior member 162.

Anterior member 162 may also be configured to include, on an external surface thereof, user controls and interface 180, which may be similar to user interface 134 of FIG. 1. That said, in some embodiments, other portions of headset 100, such as posterior members 166 or arms 164, may be configured to include user interface 180. Some of the functionalities of on-board (i.e., attached or installed, either permanently or removably) user interface 180, 134 may overlap with those of the user interface 52 of external device 150, such as, for example, enabling the receiving of user inputs to control a neurostimulation treatment or to set one or more treatment-related parameters. In some embodiments, a user may use both an on-headset user interface 180 and an on-device user interface 52, while in other embodiments only one of the two user interfaces 180, 52 is available to a user.

Electronic circuit 176 and user interface 180 may be configured to control and/or activate electrodes included in headset 100. In some embodiments, user interface 180 is configured to control and/or activate at least two, and in some embodiments more than two, pairs of electrodes. As such, in some embodiments, the stimulation circuit and/or user interface 180 are configured to enable activation of a specific electrode or of a specific pair, or channel, of electrodes, as well as adjustment of the intensity of current supplied by the activated electrodes or of other stimulation parameters of the activated electrodes and provision of user indications such as a user indication of pain, a user indication of discomfort, or a user indication of reduced or increased paresthesia. In some embodiments, any subset of the electrodes may be activated simultaneously, and in some embodiments specific subsets are predefined, for example during manufacture of the electronic circuit 176. In some such embodiments, user interface 180 enables control not only of a specific electrode or of a specific channel, but also of activated subsets of the electrodes.

In some embodiments, user controls and interface 180 includes a pair of anterior intensity buttons 181a and 181b for respectively increasing and decreasing the intensity of stimulation provided by anterior electrode systems 172, and a pair of posterior intensity buttons 182a and 182b for respectively increasing and decreasing the intensity of stimulation provided by posterior electrode systems 174. It will be appreciated that user control and interface 180 may include similar intensity buttons for each electrode included in the headset 100.

The user controls and interface 180 may further include a mode changing button 184 for activating and disabling the electronic circuit 176, as well as for changing between modes of operation of headset 100. For example, headset 100 may have multiple preset modes of operation, such as a sleep mode, a maintenance mode, and a treatment mode, and repeated operation of button 184 may switch between these modes, in addition to turning the headset on and off.

A user indication button, for example allowing the user to provide a user indication of pain, discomfort, or reduced paresthesia, may form part of user controls and interface 180 and may be disposed on an exterior surface of anterior member 162.

In some embodiments, the user controls and interface 180 may further include an audio element (not shown), such as a speaker or buzzer, for providing to the user an audible indication of use of the headset 180, such as an indication of activation of the headset, shutting down of the headset, pressing a button on interface 180, changing the stimulation mode, and the like.

As explained hereinabove, the electronic circuit and the user interface are configured to control and/or activate electrodes included in headset 100. In some embodiments, the user interface is configured to control and/or activate at least two, and in some embodiments more than two, pairs of electrodes. As such, in some embodiments, the stimulation circuit and/or the user interface are configured to enable activation of a specific electrode or of a specific pair, or channel, of electrodes, as well as adjustment of the intensity of current supplied by the activated electrodes or of other stimulation parameters of the activated electrodes. In some embodiments, any subset of the electrodes may be activated simultaneously, and in some embodiments specific subsets are predefined, for example during manufacture of the electronic circuit. In some such embodiments, the user interface enables control not only of a specific electrode or of a specific channel, but also of activated subsets of the electrodes.

In some embodiments, electronic circuit 176 includes a transceiver 196, similar to transceiver 116 of FIG. 1, which transceiver is configured to remotely communicate with external device 150.

According to embodiments of the present invention, methods for setting treatment parameters for a neurostimulation treatment session can include determining a sensory threshold based on a series of electrical stimulation pulses generated by a first plurality of electrodes, testing the reliability of the determined sensory threshold, and confirming the reliability of the determined sensory threshold. At any point in this process, i.e., after the determining, the testing, or the confirming, a first treatment threshold (recommended initial parameter) can be set based on the sensory threshold. A second treatment threshold can be set based on the first treatment threshold. The setting of a second treatment threshold based on a first treatment threshold can be based on a direct relationship between the two, for example a direct relationship derived empirically. The sensory threshold can be a treatment parameter or multiple treatment parameters at which a user indicates that he feels a paresthesia (or, more generally, any dermal sensation) related to a series of electrical stimulation pulses provided at current intensity that is monotonically increasing.

Treatment parameters, as the term is used herein can mean any (or all) of the following:
an electrical charge parameter. An electrical charge parameter can have values of total electric charge for a given time period; the given time period is typically an integer multiple of an inverse of electrical pulse frequency (which can be called the ‘period’ of the pulses. As an illustrative example, if the frequency of pulses is 50 Hz, then the inverse of the frequency is 20 milliseconds (msec), and the given time period chosen might be 1 sec. For more granular resolution, the given time period can be as short as a single ‘period’, or 20 msec in this case. The units of the electrical charge parameter can be in coulombs (a smaller division such as milli-coulombs) per second, and can be calculated by averaging or integrating delivered current (e.g., in milli-amperes) over the given time period.
a current-intensity parameter, for example a peak current or average current or steady-state current of an electrical pulse.
a pulse duration parameter and a frequency parameter, or an arithmetic combination thereof. A pulse duration, also called pulse width, is typically measured in microseconds (μsec). It can be parameterized in conjunction with the period (inverse of the frequency), for example to derive a proportion of each period in which a pulse is being applied. As an example, a pulse duration (pulse width) of 500 μsec with a 50 Hz frequency means that the pulse is being applied 0.5 msec/20 msec of the period, or 2.5% of the time.

It can be desirable to use any two or more of the foregoing electrical parameters to characterize a series of pulses. For example: current intensity and electrical charge, or current intensity and a ratio of pulse duration to an inverse of the frequency, and electrical charge and a ratio of pulse duration to an inverse of the frequency are three exemplary sets of electrical parameters that can be used to characterize a series of pulses. A skilled artisan will understand that any of the foregoing sets can be used to calculate other parameters as well: for example, if average current intensity of a pulse and the electrical charge per period are known, then the proportion of the time (out of each period) that a pulse is being applied can be calculated.

In some instances, current intensity is used herein to illustrate the use of electrical parameters in the various embodiments, for example the use of monotonically increase current intensity in a method for setting, testing, confirming or calibrating a sensory threshold. In such instances, the illustrative use of current intensity is not meant to be limiting, and, where applicable, other electrical parameters or sets of electrical parameters can be substituted for current intensity.

A sensory threshold can be measured, in an exemplary embodiment, using electrical stimulation pulses generated by anteriorly placed electrodes, e.g., electrodes 172 of FIG. 3. In other embodiments electrodes can additionally or alternatively be laterally placed, although this is not shown in FIG. 3.

The first treatment threshold, in such an embodiment, can thus be related to anteriorly-directed pulses of a treatment protocol, and can be calculated from the sensory threshold using a first empirically-developed direct relationship. The second treatment threshold in such an embodiment can be related to posteriorly-directed pulses of a treatment protocol and can be calculated from the first treatment threshold using a second empirically-developed direct relationship. In some other embodiments, the first treatment threshold can be related to posteriorly-directed pulses of a treatment protocol and the second treatment threshold to anteriorly-directed pulses of the protocol. In still other embodiments, the first and second treatment thresholds can relate, respectively, to anteriorly directed pulses and laterally directed pulses; to posteriorly directed pulses and laterally directed pulses; to laterally directed pulses and anteriorly directed pulses; to laterally directed pulses and posteriorly directed pulses; or to laterally directed pulses on opposing sides of the user's head. The first and second direct relationships can be relationships that have been derived empirically on the basis of treatment thresholds known to be in an effective range with current intensity higher than the minimum required to achieve a noticeable paresthesia and lower than a threshold that causes pain or excessive discomfort for a user. Any of the aforementioned steps can be performed using a head-mounted apparatus, including any of the headsets 100 discussed herein. According to preferred embodiments, prior to a treatment session, a user can ‘calibrate’ the initial treatment parameters, i.e., the first and second treatment thresholds set in accordance with the foregoing discussion, by increasing or decreasing a treatment parameter (e.g., current intensity or pulse frequency) based on, inter alia, user comfort. Embodiments of the method can be carried out by executing program instructions 51 stored on an external device 150 such as a mobile phone 570, the executing of the program instructions 51 being by one or more processors of the device 150, as will be described later in this disclosure.

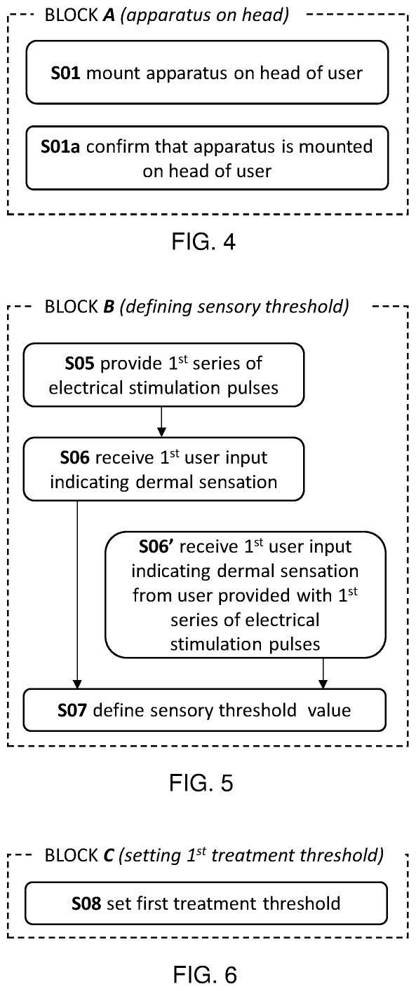

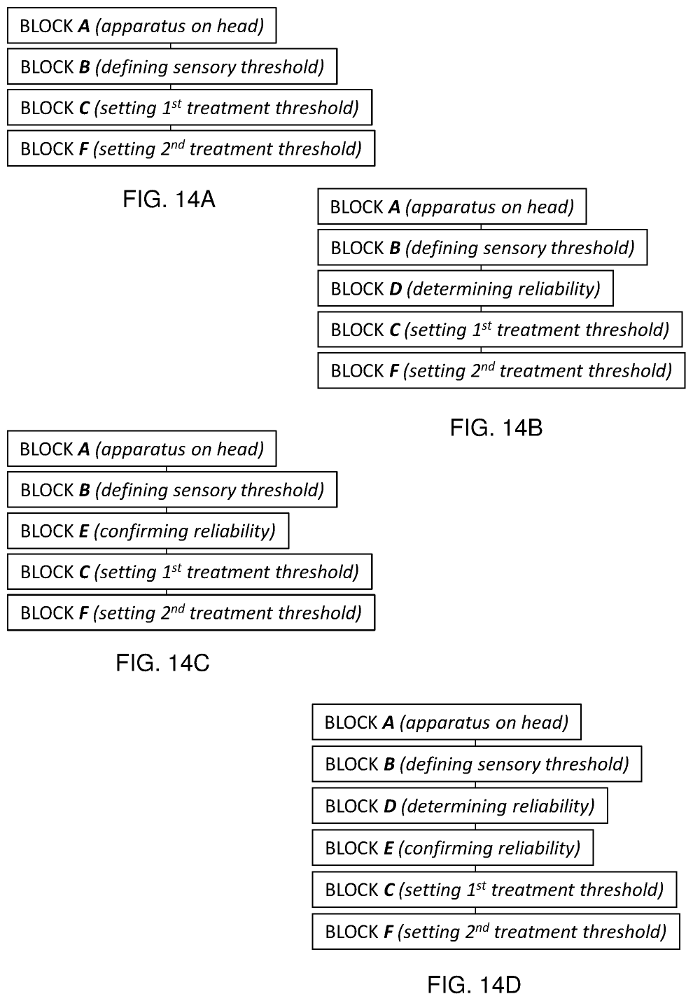

Reference is now made to FIGS. 4-9, where flowcharts of methods and method ‘building-blocks’ relating to, inter alia, defining and setting user-specific sensory and treatment thresholds are shown in accordance with various embodiments of the present invention. As seen, each building-block—a grouping of one or more method steps—is labeled with a block code (e.g., BLOCK A, etc.) so as to simplify later figures discussed hereinbelow that present different combinations of the various method building-blocks. Each building block includes a brief text description (e.g., BLOCK A (apparatus on head)) for purposes of easy and convenient identification in the flowcharts of FIGS. 14-19, where blocks are combined in various combinations. However, these brief text descriptions alongside the block designations are provided solely to enable easier comprehension of FIGS. 14-19 and are not intended to delineate or limit in any way the scope of the corresponding method steps and building blocks. Any of the individual building blocks can constitute a method according to the invention, and any combination of building blocks can jointly constitute a method according to the invention, regardless of whether such methods are explicitly illustrated and/or described herein.

Referring now to FIG. 4, ‘Block A (apparatus on head)’ comprises Step S01, which includes mounting, on the head of a user, an apparatus comprising electrodes configured to provide electrical stimulation pulses, of the type used in transdermal neurostimulation treatments. In some embodiments, the apparatus comprises the electrodes and wires connecting the electrodes to a power source. In some embodiments, the apparatus additionally comprises control circuitry. The apparatus can additionally comprise an element for holding the electrodes in place in contact with the user's scalp, such as a headband or stretchable band. In some embodiments, the apparatus can be a headset such as, for example, any of the headsets 100 discussed previously in this disclosure. When the term headset is used hereinbelow, the term should be understood to include any apparatus comprising electrodes without necessarily having some or all of the other accoutrements of a full-featured headset such as those shown in the example of headset 100 of FIG. 3. In some embodiments, Block A additionally or alternatively comprises Step S01a, which includes confirming that such an apparatus is mounted on the head of a user. As an example, if a user has not yet donned the apparatus, then most likely Step S01 will be performed. In contrast, if a user has already donned the apparatus, then most likely Step S01a will be performed. Of course, it is possible to perform both Step S01 (in which the user dons the apparatus) and Step S01a (in which the donning of the apparatus is confirmed). In some embodiments, the confirming is included in program instructions 51 carried out by one or more processors 54 of a user input device such as a mobile phone 570. In some such embodiments, the confirming can include receiving confirming information from a sensor such as, for example sensing electrodes 104. In other such embodiments, the confirming can include receiving a user input on a user interface 52 of mobile device 570. The confirming can additionally include soliciting a user input on user interface 52 prior to receiving the user input. Additionally, or alternatively, the confirming can include receiving a user input via on-board interface 180.

Block B (defining sensory threshold), as illustrated in FIG. 5, comprises a group of method steps for determining a sensory threshold for a specific user.

In Step S05, a series of electrical stimulation pulses is provided by a set of stimulating electrodes 102—in some embodiments, the set of stimulating electrodes 102 includes a plurality of anteriorly placed electrodes. The series preferably begins with faint pulses, i.e., pulses that are below a chosen electrical parameter such as a current intensity threshold or an ‘electrical charge over time threshold’ where the user experiences any dermal sensations related to the pulses. The pulses then increase monotonically in the chosen electrical parameter(s) as the series progresses. In some embodiments, other parameters of the pulses (e.g., pulse width, phase width, and frequency) remain unchanged.

Step S06 comprises receiving a user input indicating that the user has experienced a dermal sensation, e.g., paresthesia, related to the series of pulses. Alternative Step S06′ comprises receiving a user input indicating that the user has experienced, during a series of electrical stimulation pulses of increasing current intensity, or electrical charge over time, applied by a plurality of stimulating electrodes 102 to specific area or areas (e.g., anterior region) of the user's scalp, a dermal sensation related to the series of pulses. Step S06′ can be included in program instructions 51 carried out by one or more processors 54 of a user input device such as a mobile phone 570. The user input of Step S06 or Step S06′ is preferably received via a user interface 52 of mobile device 570 and/or on-board interface 180.

Note: Wherever reference numbers used in this disclosure include an apostrophe after the number, e.g., S06′, it is to indicate an alternative version of a step—such as S06—that is specifically meant to be included in program instructions carried out by one or more processors of a user input device such as a mobile phone. In each case the technical concept is the same for each of the corresponding steps in a pair such as, for example, S06 and S06′.

In response to the receiving of the first user input of Step S06 or Step S06′, a sensory threshold is defined in Step S07, where the sensory threshold for any electrical parameter or set of electrical parameters is equal to the respective parameter values at which the user input is received. In other words, the sensory threshold corresponds to the electrical parameters at which the user experiences noticeable dermal sensation (e.g., paresthesia) from the series of electrical stimulation pulses.

Referring to FIG. 6, Block C (setting 1^{st} treatment threshold) comprises Step S08, which includes setting a first treatment threshold based on a relationship between the first treatment threshold and the sensory threshold. The relationship, in preferred embodiments, is a direct relationship derived from empirical data. The first treatment threshold can be a treatment threshold for electrical stimulation pulses provided by a specific plurality of electrodes—for example electrodes 172—directed to a specific area or areas of the scalp. In some embodiments, the first treatment threshold relates to anteriorly-placed electrodes 172 which generate electrical stimulation pulses directed to anterior region(s) of the scalp, including for example portions of the forehead. The treatment threshold can comprise a value or range of values for current intensity that are deemed to provide effective transdermal neurostimulation treatment. For example, the treatment threshold can be found above a minimum value for user-perceived paresthesia and below a maximum value for user pain and/or discomfort. In some embodiments, the values for treatment threshold are determined empirically by testing users for said minimum and maximum values.

### Example 1

In an example, a direct relationship between a first treatment threshold and a sensory threshold was empirically established using a sample group of 24 users of a headset 100 for transdermal neurostimulation. The following table summarizes sensory thresholds (Tto) in milli-amperes (mA), defined in accordance with the teachings of Step S07 for each of the users; first treatment thresholds (Tt) in mA; and the ratio of Tt to Tto for each user.

| | Gender | Tto | Tt | |
|---|---|---|---|---|
| User # | (M/F) | (mA) | (mA) | Tt/Tto |
| 1 | F | 1.2 | 2.1 | 1.7 |
| 2 | F | 1.3 | 2.2 | 1.7 |
| 3 | F | 1.2 | 1.9 | 1.6 |
| 4 | F | 1.2 | 3.3 | 2.8 |
| 5 | F | 1.1 | 1.8 | 1.6 |
| 6 | F | 1.2 | 1.8 | 1.5 |
| 7 | F | 1.6 | 2.8 | 1.8 |
| 8 | F | 1.6 | 2.6 | 1.6 |
| 9 | F | 1.8 | 2.4 | 1.3 |
| 10 | F | 1.3 | 1.7 | 1.3 |
| 11 | F | 1.3 | 1.9 | 1.4 |
| 12 | F | 1.8 | 3.0 | 1.7 |
| 13 | M | 1.7 | 3.3 | 1.9 |
| 14 | M | 1.7 | 2.5 | 1.4 |
| 15 | M | 1.7 | 2.7 | 1.6 |
| 16 | M | 1.2 | 2.4 | 1.9 |
| 17 | M | 1.3 | 2.3 | 1.8 |
| 18 | M | 1.3 | 2.7 | 2.1 |
| 19 | M | 1.4 | 2.0 | 1.5 |
| 20 | M | 1.2 | 2.7 | 2.3 |
| 21 | M | 1.3 | 2.5 | 1.9 |
| 22 | M | 2.0 | 2.9 | 1.5 |
| 23 | M | 1.6 | 3.1 | 1.9 |
| 24 | M | 2.1 | 3.2 | 1.5 |

From the results for the 24 users, it can be seen that the ratio of Tt to Tto has a minimum value of 1.3, a maximum value of 2.8, and a mean of 1.7.

The first treatment threshold of Step S08 can be calculated by multiplying the defined sensory threshold for a user by a multiplier A based on the relationship between Tt and Tto as seen in the empirical data. The multiplying factor A, based on the empirical data, can be set anywhere within a range of values encompassing all or most of the users' respective preferred treatment thresholds. For example, A can be in the range of 1.0 to 3.2, inclusive; a preferred treatment threshold for all 24 users in Example 1 is found within this range. Alternatively, A can be set anywhere in the range of 1.3 to 2.8, inclusive, and once again a preferred treatment threshold for all 24 users in Example 1 is found within this range. Alternatively, A can be set anywhere in the range 1.4 to 2.0, inclusive; 19 out of 24 users have optimal treatment thresholds in this range. Yet alternatively, A can be set anywhere in the range 1.3 to 1.9; 21 out of 24 users have optimal treatment threshold in this range. Alternatively or additionally, the relationship between Tt and Tto can include other electrical parameters such as any of the electrical parameters discussed hereinabove.

Steps S07 and S08 can be included in program instructions 51 carried out by one or more processors 54 of a user input device such as a mobile phone 570.

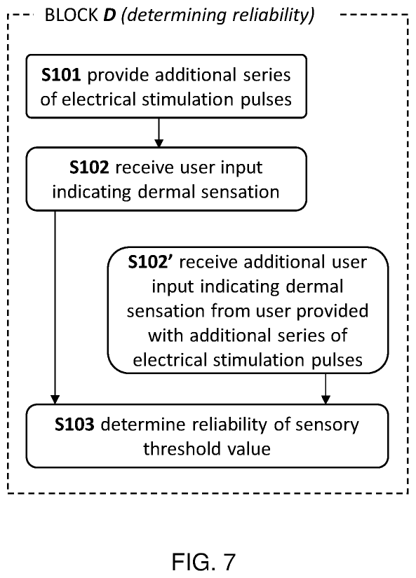

In some embodiments, it can be desirable to test the reliability of the sensory threshold, i.e., the current intensity at which the user's indication (i.e., the first user input received in Step S06 or S06′) of feeling the dermal sensation was received. Block D (determining reliability), illustrated in FIG. 7, comprises method steps directed to testing the reliability of the sensory threshold.

In Step S101, an additional series of electrical stimulation pulses is provided by a plurality of electrodes, e.g., the same plurality of electrodes used for the pulses in Step S05. Step S102 comprises receiving an additional user input indicating that the user has experienced a dermal sensation, e.g., paresthesia, related to the additional series of pulses of Step S101.

Alternative Step S102′ comprises receiving an additional user input indicating that the user has experienced, during an additional series of electrical stimulation pulses of increasing current intensity applied by a plurality of stimulating electrodes 102 to specific area or areas (e.g., anterior region) of the user's scalp, a dermal sensation related to the series of pulses. Step S102′ can be included in program instructions 51 carried out by one or more processors 54 of a user input device such as a mobile phone 570. The pulses of Step S101 or Step S102′ increase monotonically in current intensity as the series progresses, at a rate of increase that can be same as the rate of increase of the first series of pulses of Step S05 or of Step S06′, or different than that rate of increase. The user input of Step S101 or of Step S102′ is preferably received via a user interface 52 of mobile device 570 and/or on-board interface 180.

Once the additional user input has been received in Step S101 or Step S102′, reliability of the sensory threshold value can be determined in Step S103. This can include comparing the current intensity at which the additional user input is received to the sensory threshold defined in Step S07. Steps S103 and S08 can be included in program instructions 51 carried out by one or more processors 54 of a user input device such as a mobile phone 570. In some embodiments, if the additional-user-input current intensity is within ±10% of the sensory threshold defined in Step S07, the sensory threshold value can be determined to be reliable. In some embodiments, if the additional-user-input current intensity is within ±20% of the sensory threshold defined in Step S07, the sensory threshold value can be determined to be reliable. In some embodiments, if the additional-user-input current intensity is within ±30% of the sensory threshold defined in Step S07, the sensory threshold value can be determined to be reliable.

As will be discussed later, the method steps of Block D can be repeated any number of times in order to test the reliability of the sensory threshold for the user. If the sensory threshold value cannot be determined to be reliable after a desired number of repetitions, a treatment protocol based on default values can be drawn up and/or implemented. Default values can be modified based on stored user information.

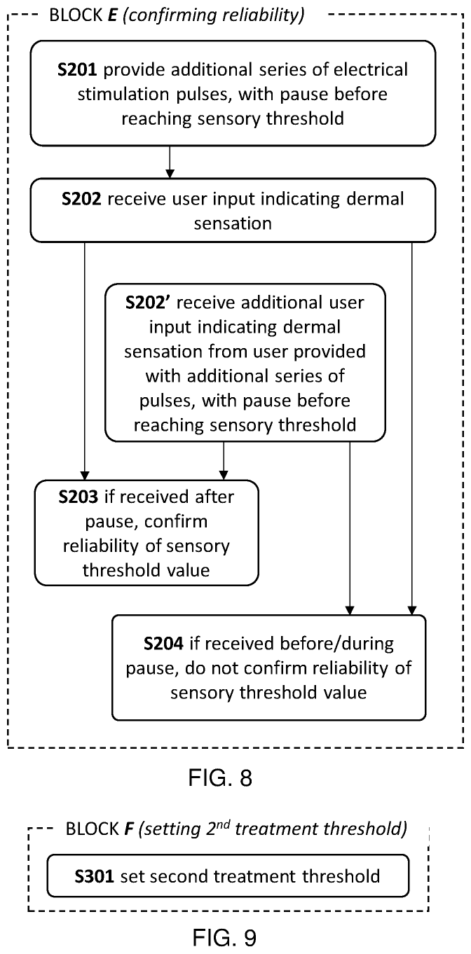

In some embodiments, it can be desirable to confirm the reliability of the sensory threshold, i.e., the current intensity at which the user's indication (i.e., the first user input received in Steps S06 or S06′) of feeling the dermal sensation was received. Block E (confirming reliability), illustrated in FIG. 8, comprises method steps directed to confirming the reliability of the sensory threshold.

In Step S201, an additional series of electrical stimulation pulses is provided by a plurality of electrodes, e.g., the same plurality of electrodes used for the pulses in Step S05. In some embodiments, the pulses can be provided by a different plurality of electrodes than that used for the pulses in Step S05. In an example, the pulses in Step S05 can be provided by an anteriorly-placed plurality of electrodes, and the pulses in Step S201 can be provided by a posteriorly-placed plurality of electrodes. As discussed hereinbelow, there can be a direct, and in some embodiments empirically-derived, relationship between respective set of treatment parameters set for anteriorly-placed plurality of electrodes and posteriorly-placed plurality of electrodes to complement the use of a different plurality of electrodes in Step S201. Step S202 comprises receiving an additional user input indicating that the user has experienced a dermal sensation, e.g., paresthesia, related to the additional series of pulses of Step S201. Alternative Step S202′ comprises receiving an additional user input indicating that the user has experienced, during an additional series of electrical stimulation pulses of increasing current intensity applied by a plurality of stimulating electrodes 102 to specific area or areas (e.g., anterior region) of the user's scalp, a dermal sensation related to the series of pulses. Step S202′ can be included in program instructions 51 carried out by one or more processors 54 of a user input device such as a mobile phone 570. The user input of Step S201 or Step S202′ is preferably received via a user interface 52 of mobile device 570 and/or on-board interface 180.

The pulses of Step S201 or Step S202′ increase monotonically in current intensity as the series progresses, at a rate of increase that can be same as the rate of increase of the first series of pulses of Steps S05 or S06′, or different than that rate of increase. In contrast to those other steps, the pulses of Step S201 or Step S202′ are interrupted by a pause before the current intensity increases to the level of the sensory threshold defined in Step S07. Preferably the pause occurs long enough before reaching the level of the sensory threshold so that the current intensity is not substantially the same as that of the sensory threshold (e.g., within 10% of the sensory threshold, within 20% of the sensory threshold, or within 30% of the sensory threshold). After the pause, which can be for less than a second, or for a second or more, or for several seconds, the series of pulses (and the increase in current intensity) continue until a user input is received in Step S202 or in Step S202′ indicating that the user has experienced the dermal sensation (e.g., paresthesia). If the user input of Steps S202 or S202′ is received after the resumption of pulses following the pause (and within an acceptable range of current intensity values from the sensory threshold), then the sensory threshold is confirmed in Step S203. On the other hand, if the user input of Steps S202 or S202′ is received before or during the pause, i.e., well before the pulses reached the sensory threshold defined in Step S07, then in Step S204 the sensory threshold is not confirmed.

Steps S203 and S204 can be included in program instructions 51 carried out by one or more processors 54 of a user input device such as a mobile phone 570.

As will be discussed later, the method steps of Block E can be performed after the steps of Block B (defining the sensory threshold) or after one or more Block D cycles/repetitions (determining the reliability of the sensory threshold). In some embodiments, it can be preferable to perform Block E before Block C (setting the first treatment threshold).

Referring now to FIG. 9, Block F (setting 2^{nd} treatment threshold) comprises Step S301, which includes setting a second treatment threshold based on a relationship between the second treatment threshold and the first treatment threshold. The relationship, in preferred embodiments, is a direct relationship derived from empirical data. The second treatment threshold can be a treatment threshold for electrical stimulation pulses provided by a specific plurality of electrodes—for example electrodes 174—directed to a specific area or areas of the scalp. In some embodiments, the second treatment threshold relates to posteriorly-placed electrodes 174 which generate electrical stimulation pulses directed to the posterior region(s) of the scalp. The treatment threshold can comprise a value or range of values for current intensity that are deemed to provide effective transdermal neurostimulation treatment. For example, the treatment threshold can be found above a minimum value for user-perceived paresthesia and below a maximum value for user pain and/or discomfort. In some embodiments, the values for treatment threshold are determined empirically by testing users for said minimum and maximum values.

### Example 2

Example 2 is an extension of Example 1 in that a direct relationship between the second treatment threshold and the first treatment threshold was empirically established using the same sample group of 24 users of a headset 100 for transdermal neurostimulation. The following table summarizes first treatment thresholds (Tt) in milli-amperes (mA), as defined in accordance with the teachings of Step S07 for each of the users; second treatment thresholds (Ot) in mA; and the ratio of Otto Tt.

| | Gender | Tt | Ot | |
|---|---|---|---|---|
| User # | (M/F) | (mA) | (mA) | Ot/Tt |
| 1 | F | 2.1 | 5.0 | 2.4 |
| 2 | F | 2.2 | 4.7 | 2.1 |
| 3 | F | 1.9 | 4.4 | 2.3 |
| 4 | F | 3.3 | 6.6 | 2.0 |
| 5 | F | 1.8 | 4.2 | 2.3 |
| 6 | F | 1.8 | 4.4 | 2.4 |
| 7 | F | 2.8 | 5.7 | 2.0 |
| 8 | F | 2.6 | 5.3 | 2.0 |
| 9 | F | 2.4 | 5.5 | 2.3 |
| 10 | F | 1.7 | 5.5 | 3.2 |
| 11 | F | 1.9 | 4.7 | 2.5 |
| 12 | F | 3.0 | 5.8 | 1.9 |
| 13 | M | 3.3 | 7.8 | 2.4 |
| 14 | M | 2.5 | 6.4 | 2.6 |
| 15 | M | 2.7 | 6.5 | 2.4 |
| 16 | M | 2.4 | 5.4 | 2.3 |
| 17 | M | 2.3 | 5.4 | 2.3 |
| 18 | M | 2.7 | 5.3 | 2.0 |
| 19 | M | 2.0 | 6.7 | 3.4 |
| 20 | M | 2.7 | 5.9 | 2.2 |
| 21 | M | 2.5 | 8.0 | 3.2 |
| 22 | M | 2.9 | 7.1 | 2.4 |
| 23 | M | 3.1 | 6.9 | 2.2 |
| 24 | M | 3.2 | 6.7 | 2.1 |

From the results for the 24 users, it can be seen that the ratio of Ot to Tt has a minimum value of 1.9, a maximum value of 3.4, and a mean of 2.4.

The second treatment threshold of Step S301 can be calculated by multiplying the first treatment threshold for a user by a multiplier B based on the relationship between Ot and Tt as seen in the empirical data. The multiplying factor B, based on the empirical data, can be set anywhere within a range of values encompassing all or most of the users' respective preferred treatment thresholds. For example, B can be in the range of 1.1 to 3.8, inclusive; a preferred treatment threshold for all 24 users in Example 2 is found within this range. Alternatively, B can be set anywhere in the range of 1.4 to 3.4, inclusive, and once again a preferred treatment threshold for all 24 users in Example 2 is found within this range. Alternatively, B can be set anywhere in the range 1.8 to 3.0, inclusive; 21 out of 24 users have optimal treatment thresholds in this range. Alternatively or additionally, the relationship between Ot and Tt can include other electrical parameters such as any of the electrical parameters discussed hereinabove.

Step S301 can be included in program instructions 51 carried out by one or more processors 54 of a user input device such as a mobile phone 570.

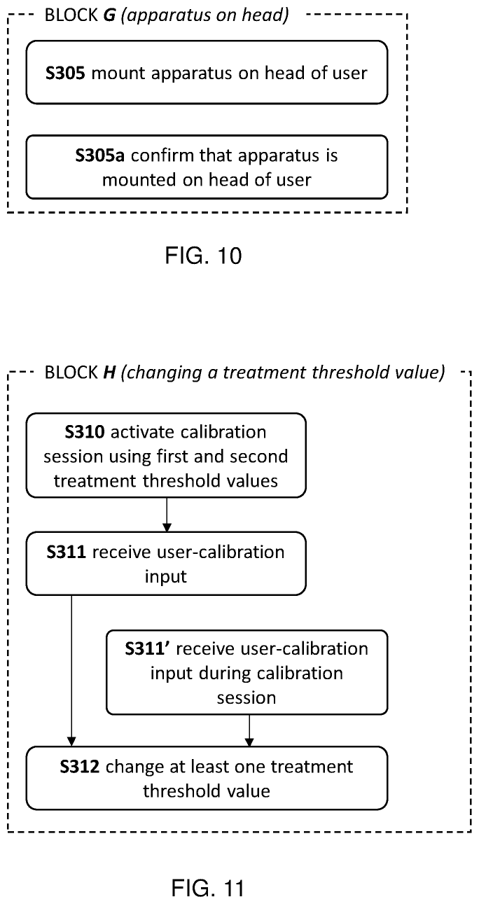

Reference is now made to FIGS. 10-13, where flowcharts of method steps and ‘building-blocks’ of method steps relating, inter alia, to calibration of treatment thresholds are shown in accordance with various embodiments of the present invention.

Referring to FIG. 10, Block G (apparatus on head) comprises Step S305, which includes mounting, on the head of a user, an apparatus comprising electrodes configured to provide electrical stimulation pulses, of the type used in transdermal neurostimulation treatments. In embodiments, the apparatus can be a headset such as, for example, any of the headsets 100 discussed previously in this disclosure. The apparatus can be any of the apparatuses described with respect to FIG. 4 and the discussion of Block A earlier in the disclosure. The apparatus can be the same apparatus used in Block A, and may still be in place since the performance of Block A steps (and/or any other method steps). In some embodiments, Block G additionally or alternatively comprises Step S305a, which includes confirming that such an apparatus is mounted on the head of a user. In some embodiments, the confirming is included in program instructions 51 carried out by one or more processors 54 of a user input device such as a mobile phone 570. In some such embodiments, the confirming can include receiving confirming information from a sensor such as, for example sensing electrodes 104. In other such embodiments, the confirming can include receiving a user input on a user interface 52 of mobile device 570. The confirming can additionally include soliciting a user input on user interface 52 prior to receiving the user input. Additionally, or alternatively, the confirming can include receiving a user input via on-board interface 180.

In some embodiments, it can be desirable to have a user calibrate the treatment thresholds set in Steps S08 and S301 in a dedicated calibration session. The calibration by the user can include changing a treatment protocol parameter—for example the current intensity—in order to increase the comfort level of undergoing a treatment. In some cases, a user might prefer to increase the current intensity, and in other cases she might prefer to decrease the current intensity or modify another electrical parameter. For example, decreasing the electrical charge over time might include increasing the current intensity by 5% but shortening the duration of the pulses by 10%. In other examples a user might wish to make the electrical stimulation pulses of the transdermal neurostimulation treatment shorter or longer, and/or more frequent or infrequent. In some embodiments, the system 101 can be configured to receive such calibration-inputs from a user and, in response, make the requested changes very quickly (e.g., within less than a second after the user finishes making changes, or less than 500 milliseconds, or less than 200 milliseconds). In some embodiments, the system 101 can be configured to adjust—in response to the user-calibration inputs—not only the value of the parameter directly changed by the user, but also the value of at least one additional parameter. For example, if a user changes the value of a current intensity treatment protocol parameter, the system 101 may be configured to adjust, for example, one or more of pulse width (duration), phase width, and/or frequency. The at one or more additional parameter may be automatically changed in response to the user-calibration input, in order to serve a treatment goal. For example, it can be a treatment goal to maintain an overall average current such that the overall average current during a treatment stays at a predetermined value. (One way of calculating average current is to multiply current intensity by the ratio of phase width to pulse width, with average current typically being measured in milliamperes.) Thus, decreasing current intensity could trigger an increase in phase width. As another example, it can be a treatment goal to maintain average current within an acceptable variance (e.g., ±30%, ±20% or +10%), of a predetermined value. In both of these examples, the predetermined value can be the overall average current value before the user-calibration inputs.

In some embodiments, a dedicated user calibration session can take place immediately before a treatment session, and in other embodiments the calibration session can be a standalone interaction with the system 101 and its components such as headset 100, with mobile device 570 serving as user-input device by executing, on command program instructions 51 by the one or more processors 54. In some embodiments, a calibration session can take place immediately or soon after the performance of Step S301 (setting the second treatment threshold).

Referring now to FIG. 11, Block H (changing a treatment threshold value) comprises a group of method steps related to user calibration of treatment thresholds.

In Step S310, a calibration session is activated; in some embodiments the base (initial) treatment threshold values are those set in Steps S08 and S301 and in some other embodiments one or both of the initial treatment threshold values has already been changed in a previous calibration session. A calibration can be initiated by a user, or it can be initiated by the system running an app (executing program instructions 51) on a user input device (e.g., mobile device 570). According to embodiments, electrical stimulation pulses are delivered to two portions of the user's scalp (e.g., the anterior and posterior portions) during the calibration session.

Step S311 comprises receiving a user-calibration input from the user, where a user-initiated change can be made to the value of any treatment parameter as discussed above. Alternative Step S311′ comprises receiving a user-calibration input after the activation of a calibration session using first and second current-intensity treatment thresholds and/or other electrical parameter thresholds as respective initial current intensities applied to two portions of the user's scalp (e.g., the anterior and posterior portions), or, equivalently, using current-intensity treatment thresholds that have been previously changed by the user in an earlier calibration session. All user inputs, sensory thresholds, treatment thresholds, user-calibration inputs and other data related to and/or generated by the system 101 can be stored in any one of the external devices 150 as well as on external servers in the cloud (not shown). Step S311′ can be included in program instructions 51 carried out by one or more processors 54 of a user input device such as a mobile phone 570. The user-calibration inputs of Step S311 or Step S311′ are preferably received via a user interface 52 of mobile device 570 and/or on-board interface 180.

In response to receiving a user-calibration input during a treatment session (Step S311 or S311′), a change is effected, in Step S312, to at least one treatment threshold value. As discussed earlier, one or more other parameters may be modified so as to maintain treatment efficacy, for example by keeping overall average current within a predetermined range. Step S312 can be included in program instructions 51 carried out by one or more processors 54 of a user input device such as a mobile phone 570.

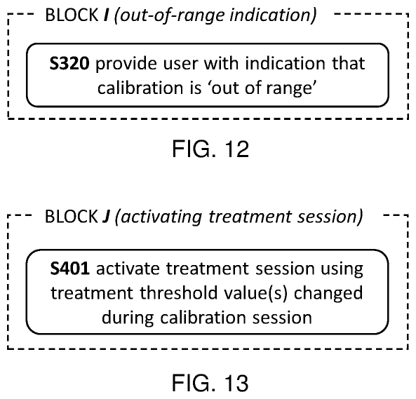

We now refer to FIG. 12, which shows method-step Block I (out-of-range indication) comprising Step S320. In some embodiments, Step S320 alternatively or additionally includes, in response to receiving a user-calibration input in Step S311 or S311′, providing the user with an indication that the change request embodied in a calibration-user input received during the calibration session will take the treatment parameters outside of the recommended range for effective neurostimulation treatment. A user receiving such an indication, e.g. through the user interface 52 of mobile device 570 can be given an opportunity to ‘correct’ the calibration-user input so as to remain within the recommend range of parameter values. Step S320 can be included in program instructions 51 carried out by one or more processors 54 of a user input device such as a mobile phone 570.

Block J (activating treatment session), illustrated in FIG. 13, comprises Step S401, which involves activating a transdermal neurostimulation treatment session using one or more treatment parameters changed in a calibration session.

Reference is made to FIGS. 14-17, which illustrate examples of methods, according to embodiments, that include method steps building blocks discussed with reference to FIGS. 4-13.

FIGS. 14A-D show several options for setting treatment thresholds in accordance with the various embodiments.

In FIG. 14A, an exemplary method is shown to include mounting a headset/confirming that the headset is mounted (Block A), defining a sensory threshold (Block B), setting a first treatment threshold (Block C) and setting a second treatment threshold (Block F).

In FIG. 14B, another exemplary method is shown to include mounting a headset/confirming that the headset is mounted (Block A), defining a sensory threshold (Block B), determining the reliability of the sensory threshold (Block D), setting a first treatment threshold (Block C) and setting a second treatment threshold (Block F).

In FIG. 14C, yet another exemplary method is shown to include mounting a headset/confirming that the headset is mounted (Block A), defining a sensory threshold (Block B), confirming the reliability of the sensory threshold (Block E), setting a first treatment threshold (Block C) and setting a second treatment threshold (Block F).

In FIG. 14D, yet another exemplary method is shown to include mounting a headset/confirming that the headset is mounted (Block A), defining a sensory threshold (Block B), determining the reliability of the sensory threshold (Block D), confirming the reliability of the sensory threshold (Block E), setting a first treatment threshold (Block C) and setting a second treatment threshold (Block F).

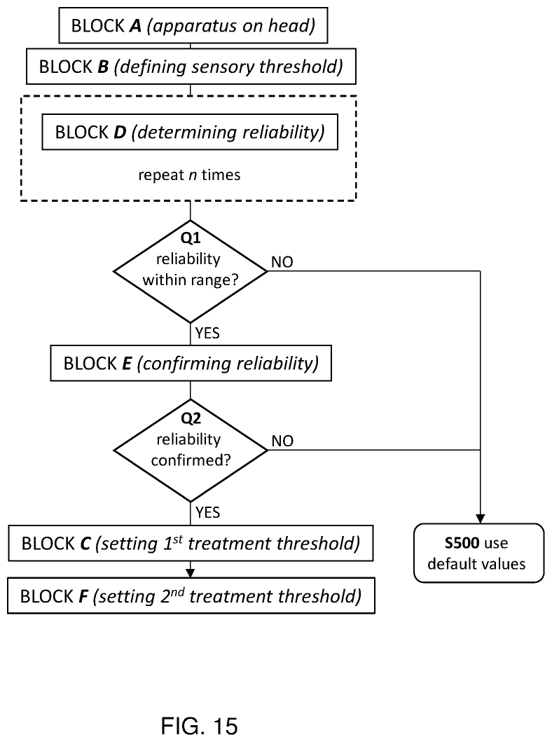

FIG. 15 illustrates another exemplary method, that includes mounting a headset/confirming that the headset is mounted (Block A); defining a sensory threshold (Block B); and determining the reliability of the sensory threshold (Block D), with the Block D method steps repeated n times, with n being a integer greater than 1 (n is not greater than 10 in some embodiments, not greater than 8 in other embodiments, and not greater than 6 in still other embodiments). If after n repetitions, the reliability is not determined in Step S103 to be within a predetermined range, e.g., the sensory threshold is repeated with accuracy of ±30%, ±20% or ±10% (branch Q1), then Step S500 is carried out, which involves using a set of system default values as initial (pre-calibration) treatment threshold values. If Q1 is resolved positively, then the method of FIG. 15 continues with confirming the reliability of the sensory threshold (Block E). If the reliability cannot be confirmed (i.e., Step S204 has been carried out) then Step S500 (reverting to default initial treatment threshold values) is performed. If Q2 is resolved positively, then performing the method additionally includes carrying out the method steps of setting a first treatment threshold (Block C) and setting a second treatment threshold (Block F).

In some embodiments, other combinations of the disclosed method steps and method-step building blocks may be performed, and all of them are within the scope of the present invention. It is mentioned for the sake of clarity that the method steps of Block A can be carried out whenever there is a discontinuity in the performance of any of the exemplary methods.

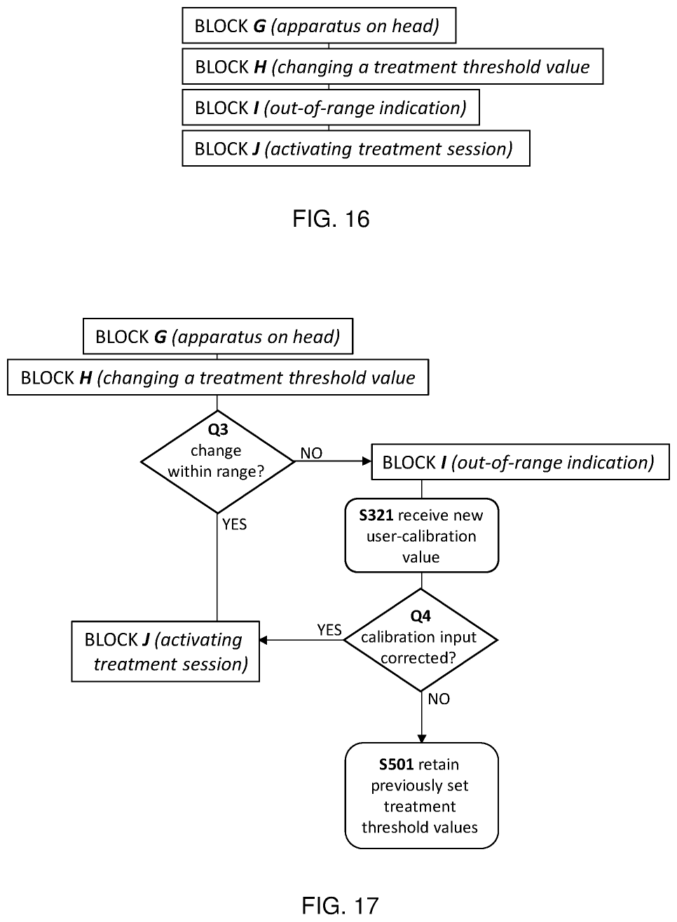

FIGS. 16-17 illustrate options for completing calibration of treatment thresholds in accordance with the various embodiments.

In FIG. 16, an exemplary method is shown to include mounting a headset/confirming that the headset is mounted (Block G), changing at least one treatment threshold value in a calibration session (Block II), indicating that a user-calibration input would take values out of range (Block I), and activating treatment session based on one or more values changed in a calibration session (Block J). Not all steps and blocks of the method need be performed in all embodiments. For example, in some embodiments Block G is performed only if the method is not performed consecutively following one of the methods of FIGS. 13-15 (or equivalent) in which the headset is already in place. Block I is performed only if a received user-calibration input would take the treatment parameters out of the desired range. Block J is optional, as a calibration session can be performed without continuing directly into an actual transdermal neurostimulation session.

FIG. 17 illustrates another exemplary method, that optionally includes mounting a headset/confirming that the headset is mounted (Block G), and includes changing at least one treatment threshold value in a calibration session (Block II). If (in Q3) a received input for a change is out of range, then Block I is carried out. If (in Q3) no received input for a change is out of range, then the method continues and Block J (activating an actual treatment session with at least one changed treatment parameter) can optionally be carried out. If Block I is invoked in branching at Q3, then Step S321, receiving a new user-calibration value, can be performed. If (at Q4) a new or ‘correct’ (in range) user-calibration input has been received in Step S321, then Block J (activating an actual treatment session with at least one changed treatment parameter) can optionally be carried out; otherwise (i.e., if it is determined at Q4) that a new/correct/in-range user-calibration has not been received, then the system defaults, in Step S501 to retaining previously-set treatment threshold values.

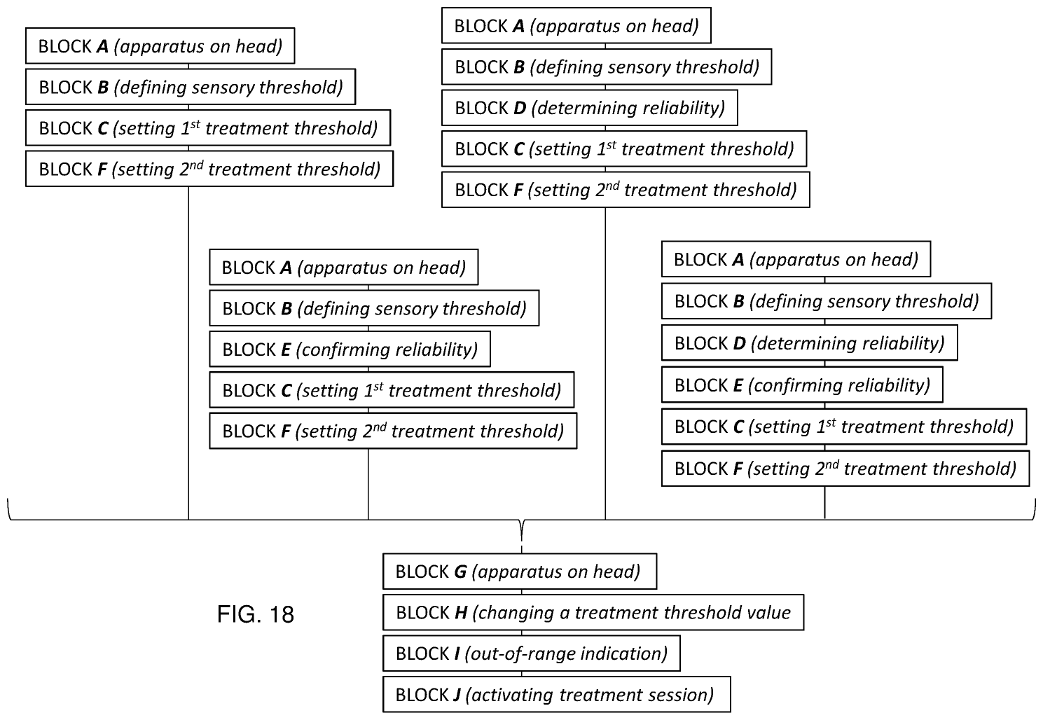

FIG. 18 illustrates, schematically, another exemplary method, in which any one of the four methods described with reference to FIGS. 14A-D, respectively, can be combined ‘end-to-end’ with the method of FIG. 16.

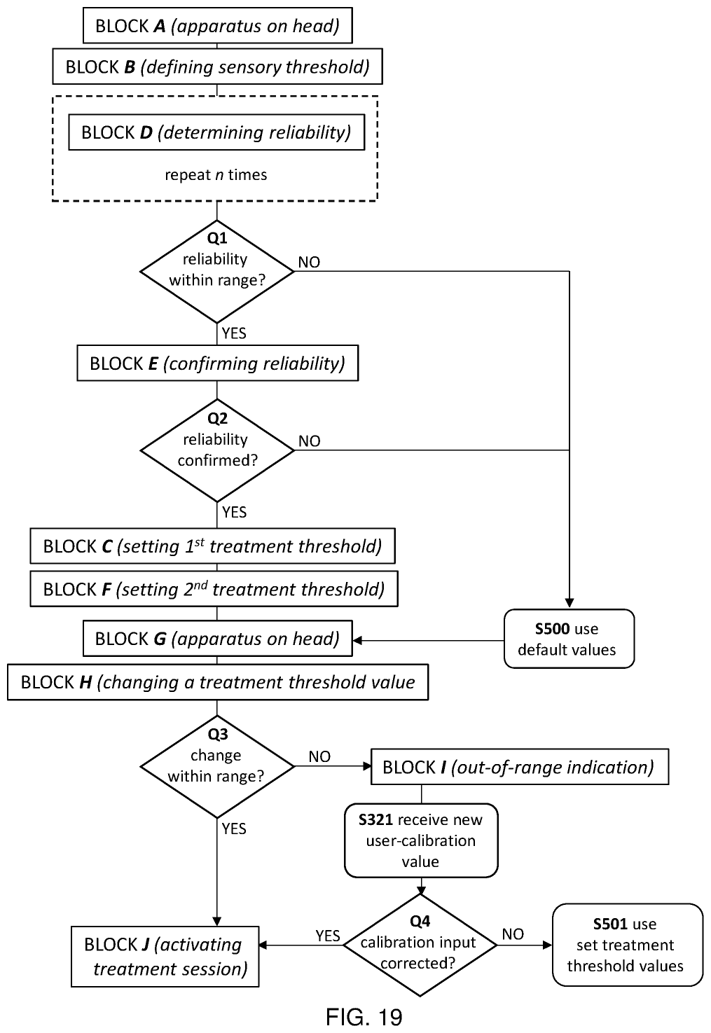

FIG. 19 illustrates, also schematically, another exemplary method, in which the method of FIG. 15 can be combined ‘end-to-end’ with the method of FIG. 17.

Any of the methods illustrated in FIGS. 13-19, or their equivalent, can be included in program instructions 51 (e.g., for an app) for execution by one or more processors 54 of a mobile device 570.

It will be obvious to the skilled artisan that the examples and embodiments described herein are not limited to a ‘first set the anterior threshold, then set the posterior threshold’ paradigm as has been employed for the sake of convenience throughout much of this disclosure.

As an example, a sensory threshold may be determined using a posteriorly-directed set of electrodes, that sensory threshold then ‘translated’ to a posterior treatment threshold, and that posterior treatment threshold ‘translated’ to an anterior treatment threshold, both ‘translations’ being based on an empirically-derived relationship between the respective current intensity thresholds.

As another example, any number of different cranial or peripheral nerves targeted for transdermal neurostimulation treatment can be addressed in a desired order, and treatment thresholds may be set in accordance with empirically-derived relationships between the various regions or nerves (or nerve groups) in that desired order. In some embodiments, the targeted nerve branches can be laterally located on the left and right sides of the user's head.

Following are examples of the order in which treatment thresholds can be determined for different nerve branches or regions of the user's scalp, for two pluralities of electrodes targeting two regions of the scalp (A=anterior; P=posterior; LL=left lateral; RL=right lateral): A-P; A-LL; A-RL; P-A; P-LL; -RL; LL-A; LL-P; LL-RL; RL-A; RL-P; and RL-LL. Obviously when three or more regions of the scalp are targeted and corresponding numbers of electrodes are used, the number of possible combinations increases accordingly.

It should also be clear that the embodiments are not limited to a user's head, and may be applied to any limb or region of the body for which empirical relationships can be derived between sensory thresholds and treatment thresholds, and further between different treatment thresholds.

Any such variations applied to the methods and devices disclosed herein are clearly within the scope of the present invention.

As used herein, wherever it is written that a given method step or a given group of method steps can be ‘included’ in program instructions, it should be understood that instructions to perform the given method step or given group of method steps can be included in said program instructions, such that execution of said program instructions (e.g., by one or more computer processors or one or more processors of a mobile device, as appropriate) causes the one or more processors to perform the given method step or given group of method steps, or causes the one or more processors to cause the given method step or given group of method steps to be carried out, as appropriate.

As used herein in the specification and in the claims section that follows, the term “or” is considered as inclusive, and therefore the phrase “A or B” means any of the groups “A”, “B”, and “A and B”.

As used herein in the specification and in the claims section that follows, the term “pulse” relates to an electrical signal, for example applied via an electrode or sensed by an electrode.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. Similarly, the content of a claim depending from one or more particular claims may generally depend from the other, unspecified claims, or be combined with the content thereof, absent any specific, manifest incompatibility therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining user-specific treatment parameter values for a multi-channel transdermal neurostimulation treatment applied at multiple locations on a user's scalp, the method comprising:
a. setting a first set of treatment parameter values for a first electrode-array in engagement with a first portion of the user's scalp for administering electrical pulses thereto, according to a first correlation that includes a first direct relationship between at least one parameter value of said first set of treatment parameter values and a corresponding at least one parameter value of a set of user-specific sensory-threshold parameter values; and
b. setting a second set of treatment parameter values for a second electrode-array in engagement with a second portion of the user's scalp for administering electrical pulses thereto, according to a second correlation that includes a second direct relationship between at least one parameter value of said second set of treatment parameter values and said at least one parameter value of said first set of treatment parameter values.

2. The method of claim 1, wherein each respective set of treatment parameter values includes values for at least two of:
i. an electrical charge parameter having values of total electric charge for a given time period, where the given time period is an integer multiple of an inverse of electrical pulse frequency,
ii. a current-intensity parameter, and
iii. a pulse duration parameter and a frequency parameter, or an arithmetic combination thereof.

3. The method of either one of claim 1 or 2, wherein either (i) said first portion of the scalp is an anterior portion and said second portion of the scalp is a posterior portion, or (ii) said first portion of the scalp is a posterior portion and said second portion of the scalp is an anterior portion.

4. The method of any preceding claim, wherein said second direct relationship is based in part on a relationship between respective electrode surface-areas of said first and second electrode arrays.

5. The method of any preceding claim, wherein values of the set of user-specific sensory-threshold parameter values are based on a first user input received during application of a series of electrical pulses to a single one of the first and second portions of the user's scalp.

6. The method of any preceding claim, wherein said first and second correlations are linear relationships based on respective empirical correlations.

7. The method of any preceding claim, wherein according to said first direct relationship, an electrical charge parameter value of said first set of treatment parameter values is equal to an electrical charge parameter value of said set of user-specific sensory-threshold parameter values multiplied by A, where A is not less than 1.3 and not greater than 1.9.

8. The method of any one of claims 1 to 6, wherein according to said second direct relationship, an electrical charge parameter value of said second set of treatment parameter values is equal to an electrical charge parameter value of said first set of treatment parameter values multiplied by B, where B is not less than 1.1 and not greater than 3.8.

9. The method of any preceding claim, additionally comprising:
calibrating treatment thresholds, wherein the calibrating includes:
i. activating a transdermal neurostimulation calibration session using said first and second set of treatment parameter values as respective treatment parameter values applied to said first and second portions of the user's scalp,
ii. receiving a user-calibration input during said calibration session, and
iii. in response to receiving said user-calibration input, changing at least one value of said first and second set of treatment parameter values.

10. The method of claim 9, wherein said changing of said at least one value of said first and second set of treatment parameter values includes modifying at least one treatment parameter value that is not a current intensity.

11. The method of either one of claim 9 or 10, wherein said changing is constrained such that an electrical charge parameter value is constrained to being within a predetermined range.

12. The method of any one of claims 9 to 11, wherein said changing is constrained such that the number of changes per calibration session is limited to a predetermined number of changes.

13. The method of any one of claims 9 to 12, wherein said changing is constrained to a predetermined interval of time.

14. The method of any one of claims 9 to 13, wherein the time between receiving of the user-calibration input and completing the changing is less than one second.

15. The method of any one of the preceding claims, wherein the transdermal neurostimulation treatment includes anteriorly-applied electrical pulses targeting the trigeminal nerve and posteriorly-applied electrical pulses targeting the occipital nerve.

16. The method of any preceding claim, wherein said administering of electrical pulses is by an apparatus including electrodes and mounted on the user's head, said apparatus comprising a headset.

17. The method of claim 16, wherein at least one of said first user input and said user-calibration input is received via a computer software program running on an external user-operated device in electronic communication with said headset.

18. The method of claim 16, wherein (i) said headset comprises a on-headset user interface attached to said headset, and (b) at least one of said first user input and said user-calibration input is received via said on-headset user interface.

19. A method of determining user-specific treatment parameter values for a multi-channel transdermal neurostimulation treatment applied at multiple locations on a user's scalp, the method comprising:
a. setting a first set of treatment parameter values for a first electrode-array in engagement with a first portion of the user's scalp for administering electrical pulses thereto; and
b. setting a second set of treatment parameter values for a second electrode-array in engagement with a second portion of the user's scalp for administering electrical pulses thereto, according to a second correlation that includes a direct relationship between at least one parameter value of said second set of treatment parameter values and said at least one parameter value of said first set of treatment parameter values.

20. The method of claim 19, wherein each respective set of treatment parameter values includes values for at least two of:
i. an electrical charge parameter having values of total electric charge for a given time period, where the given time period is an integer multiple of an inverse of electrical pulse frequency,
ii. a current-intensity parameter, and
iii. a pulse duration parameter and a frequency parameter, or an arithmetic combination thereof.

21. The method of either one of claim 19 or 20, wherein either (i) said first portion of the scalp is an anterior portion and said second portion of the scalp is a posterior portion, or (ii) said first portion of the scalp is a posterior portion and said second portion of the scalp is an anterior portion.

22. The method of any one of claims 19 to 21, wherein said direct relationship is based in part on a relationship between respective electrode surface-areas of said first and second electrode arrays.

23. The method of any one of claims 19 to 22, wherein said correlation is a linear relationship based on an empirical correlation.

24. The method of any one of claims 19 to 23, wherein according to said direct relationship, an electrical charge parameter value of said second set of treatment parameter values is equal to an electrical charge parameter value of said first set of treatment parameter values multiplied by B, where B is not less than 1.1 and not greater than 3.8.

25. The method of any one of claims 19 to 24, wherein the transdermal neurostimulation treatment includes anteriorly-applied electrical pulses targeting the trigeminal nerve and posteriorly-applied electrical pulses targeting the occipital nerve.

26. The method of any one of claims 19 to 25, wherein said administering of electrical pulses is by an apparatus including electrodes and mounted on the user's head, said apparatus comprising a headset.

27. A method of determining user-specific treatment parameters for a multi-channel transdermal neurostimulation treatment applied at multiple locations on a user's scalp, the method comprising:
a. providing, to the user's scalp, a first series of electrical pulses having electrical parameter values that increase at a first rate of increase, wherein said respective electrical parameter values comprise at least one of an electrical charge parameter value and a current-intensity value;
b. responsively to receiving a first user input indicating that the user has felt a dermal sensation related to the application of said first series of electrical pulses, defining, as a set of sensory-threshold parameter values, a plurality of respective electrical parameter values at which said first user input is received;
c. providing, to the user's scalp, a reliability-confirming series of electrical pulses having respective electrical parameter values that increase at a second rate of increase, wherein said respective electrical parameter values comprise at least one of an electrical charge parameter value and a current-intensity value, the providing being such that said reliability-confirming series is (i) paused before respective electrical parameter values reach said sensory-threshold parameter values, and (ii) subsequently resumed after a pause;
d. responsively to receiving, after said pause and at respective electrical parameter values within a predetermined range of values surrounding said sensory-threshold parameter values, a second user input indicating that the user has felt a dermal sensation related to the application of said reliability-confirming series of electrical pulses: confirming the reliability of said sensory-threshold parameter values; and
e. applying the multi-channel transdermal neurostimulation treatment using treatment parameter values based on said reliability-confirmed sensory-threshold parameter values.

28. The method of claim 27, wherein said treatment parameter values include values for at least two of:
i. an electrical charge parameter having values of total electric charge for a given time period, where the given time period is an integer multiple of an inverse of electrical pulse frequency,
ii. a current-intensity parameter, and
iii. a pulse duration parameter and a frequency parameter, or an arithmetic combination thereof.

29. The method of either one of claim 27 or 28, wherein said first series of electrical pulses is applied to a first portion of the user's scalp, and said reliability-confirming series of electrical pulses is applied to a second portion of the scalp that is different from the first portion.

30. The method of either one of claim 27 or 28, wherein said first series of electrical pulses and said reliability-confirming series of electrical pulses are both applied to a first portion of the user's scalp.

31. The method of any one of claims 27 to 30, wherein either (i) said first portion of the scalp is an anterior portion and said second portion of the scalp is a posterior portion, or (ii) said first portion of the scalp is a posterior portion and said second portion of the scalp is an anterior portion.

32. The method of any one of claims 27 to 31, wherein the transdermal neurostimulation treatment includes anteriorly-applied electrical pulses targeting the trigeminal nerve and posteriorly-applied electrical pulses targeting the occipital nerve.

33. The method of any one of claims 27 to 32, wherein said providing of said first series of electrical pulses and said providing of said reliability-confirming series of electrical pulses are by an apparatus including electrodes and mounted on the user's head, said apparatus comprising a headset.

34. The method of claim 33, wherein at least one of said first user input and said second input is received via a computer software program running on an external user-operated device in electronic communication with said headset.

35. The method of claim 34, wherein (i) said headset comprises a on-headset user interface attached to said headset, and (ii) at least one of said first user input and said second user input is received via said on-headset user interface.
